(19) United States
(12) Patent Application Publication
(10) Pub. No.: US 2014/0187851 A1
(43) Pub. Date: Jul. 3, 2014

(54) APPARATUS FOR THERAPEUTIC TREATMENT WITH PULSED RESONANT ELECTROMAGNETIC WAVES

(71) Applicant: Bruno Massimo Cetroni, Novate Mezzola (Sondrio) (IT)

(72) Inventor: Bruno Massimo Cetroni, Novate Mezzola (Sondrio) (IT)

(73) Assignee: THERESON S.p.A., Milano (IT)

(21) Appl. No.: 14/125,927

(22) PCT Filed: Jun. 14, 2012
(86) PCT No.: PCT/IB2012/053006

§ 371 Date: Mar. 14, 2014

(30) Foreign Application Priority Data
Jun. 15, 2011 (IT) TO2011A000527

(51) Int. Cl. A61N 2/02

(52) U.S. Cl. CPC A61N 2/02
USPC 600/14

(57) ABSTRACT

An apparatus for therapeutic treatment with electromagnetic waves comprising a control circuit configured for generating a signal to be transmitted to an antenna for the generation of electromagnetic waves. The signal comprises a plurality of base pulses grouped in pulse packets and pulse trains, where each pulse packet consists of a series of base pulses followed by a first pause, and where each pulse train consists of a series of pulse packets followed by a second pause. In particular, the control circuit is configured for reversing the polarity of the base pulses after a given time interval.

### TEXT OF THE DESCRIPTION

1. Field of the Invention

The present invention relates to an apparatus for therapeutic treatment with pulsed resonant electromagnetic waves.

The present invention has been developed for the therapy of tissue lesions and other pathological conditions, and is based upon the reparative stimulus of the tissues caused by magnetic resonance at a biological level.

2. Description of the Known Art

Therapies of the electromagnetic category involve various methods for applying an electrical or electromagnetic field to an ulcer or to a tissue lesion of some other kind in order to facilitate cell growth and proliferation of new tissue. For example, the application of external electrical or electromagnetic fields has become a standard in the therapy of bone fractures, but is increasingly present also in the treatment of soft-tissue lesions.

Clinical research has shown that treatment with electrical or electromagnetic stimuli can accelerate healing of skin ulcers that do not respond to more conventional therapies. For example, stimuli with pulsating electrical field have shown a certain clinical effectiveness in the treatment of bed sores. This therapeutic approach is based upon the observation, initiated approximately 60 years ago, that the electrical potentials on ulcers are negative up to healing and on the related hypothesis that living tissues possess surface potentials that regulate the proliferative phase of the cells. Hence, tissue repair can be induced/stimulated by the application of a negative potential. Even though this approach proves simplistic and antiquated as compared to in-depth studies on the healing mechanisms, there is in any case a certain scientific evidence that the electromagnetic stimulus activates the macrophages and increases cell proliferation, collagen synthesis, and the expression of fibroblast receptors for transforming growth factor beta.

There are currently in use or have been used in the past various methods that exploit emission of physical energy for stimulating tissue repair, in particular of skin ulcers. Many of these methods involve the use of electric currents for stimulating tissue growth. Other devices envisage an antenna for applying electromagnetic energy at radiofrequency through the body for therapeutic purposes. A large category of devices likewise envisages the use of electromagnetic emitters such as solenoids, which are applied, according to the shape, either in the proximity or around the anatomic part to be treated, so as to subject the tissues to be treated to a local electromagnetic stimulus. All these devices produce, for the characteristics of intensity and frequency of the electromagnetic stimulus of a continuous type, a thermal effect within the tissue that should act as stimulus to regeneration.

A subcategory of electromagnetic devices uses, instead, a pulsed signal so as to generate a stimulus with the minimum passage/generation of thermal energy. Examples of these technologies are Diapulse and Dermagen, used for the treatment of ulcers and lesions via the application of low-frequency pulsed electromagnetic fields. At the state of the art, these devices have not provided scientific irrefutable proof of clinical effectiveness even though the studies seem to agree on a certain improvement in healing times. On the other hand, their presupposed mechanism of operation at a cellular level has never been explained.

The document No. US-A-2006/0129189 describes an apparatus for the treatment of chronic lesions using electromagnetic energy. The apparatus comprises a generator of electromagnetic energy configured for producing high-frequency pulses, with frequencies that range from 1 to 1000 MHz. The generator is coupled to applicators that apply to the areas to be treated treatment energies in the region of 1-300 mW/cm². This document also describes treatment devices that envisage the passage of electric currents in windings of wires so as to create magnetic fields; in this case, the frequency of the electrical pulses is relatively low, typically in the range of low frequencies or audible frequency.

The document U.S. Pat. No. 5,584,863 describes a system for modifying growth and repair of cells and tissues by application of pulsed electromagnetic fields with frequencies of the order of megahertz. Use of bursts of sinusoidal pulses or pulses with other waveforms is described, with each burst of pulses that contains from 1100 to 10000 pulses per burst and a frequency of repetition of the bursts comprised between 0.01 and 1000 Hz.

Finally, the document No. EP 1 723 958 describes an apparatus for generating magnetic fields in the range between 0.3 Hz and 1 kHz. In particular, in one embodiment, the apparatus generates a pulse sequence, where each pulse is followed by a brief pause so that groups of pulses are generated. The duration of the pulses is typically between 0.5 and 150 ms, preferably approximately 2 ms, whilst the pauses have a duration that is less than 10 s. The document mentions the fact that with this scheme it is possible to generate signals with specific contributions in the spectrum, in particular between 0.3 Hz and 1 kHz. However, the document does not provide clear indications on selection of the time characteristics, and devotes above all attention to a specific waveform of the base pulse.

### OBJECT AND SUMMARY OF THE INVENTION

The inventor has noted, however, that in the known art there do not exist systems that, in a specific way, subject the various types of biological tissue and the various organs implicated by tissue damage and by consequent repair to a stimulus that will set the cell structures of said tissues and organs in magnetic resonance in order to produce a stimulus for the repair of tissue lesions.

In fact, experiments have shown that different tissues and organs respond, in vivo, to frequencies of weak electromagnetic fields that have the property of sending specific cell structures of those organs into resonance.

These “characteristic” frequencies are, for example, those of the electroencephalogram, which have a frequency range that falls between 0.1 and 42 Hz. Likewise, also other organs, tissues, and cell structures have typical frequency ranges, which have the property of responding to “resonance stimuli”, if exposed to magnetic fields pulsating at these frequencies.

Consequently, in order to determine a positive stimulus to tissue repair it is necessary to obtain resonance effects from the various organs/tissues involved simultaneously. There is then posed the far from simple problem of generating in the anatomical part involved a pulsating magnetic field that possesses the frequency contents of the various characteristic ranges of each tissue (in general different from, but at times superimposable on, the typical band from 0.1 to 100 Hz) in a targeted way.

Hence, unlike the observations of the document No. EP 1 723 958, it seems that it is not necessary to stimulate the target in the range between 0.3 Hz and 1 kHz, avoiding specific frequencies, such as for example the frequencies around 50 Hz, but stimulating in a targeted manner the characteristic frequencies of the target in the range between 0.1 Hz and 100 Hz, above all in the range between 0.1 Hz and 25.9 Hz.

In fact, the inventor has found that pulsating electromagnetic fields that have different or wider frequency ranges can present a low probability of inducing the effect of therapeutic resonance. Furthermore, said electromagnetic fields will certainly provide frequencies that cause at the most a thermal or saturating stimulus also by virtue of the fact that the amplitudes typically used in the known art are much higher than the useful ones, frequently causing an exchange of energy that can instead be harmful.

To be able to obtain a waveform that is adequate for creating simultaneously clearly defined series of frequencies only within certain ranges, it is hence necessary to “construct” a wave with specific frequency contributions.

The object of the present invention is to provide an apparatus for therapeutic treatment that will enable generation of an effect of electromagnetic resonance at the level of the cell, metabolic and tissue structures of the organism that is capable of producing a stimulus on the biological mechanisms that are at the basis of tissue regeneration.

According to the present invention, said object is achieved by an apparatus having the characteristics forming the subject of claim 1.

The claims form an integral part of the teaching provided herein in relation to the invention.

As mentioned previously, unlike the apparatuses according to the known art, the present invention exploits in a deterministic way the property of cell structures to go into electromagnetic resonance when they are subjected to coherent and structured signals in a specific way to obtain this effect.

In particular, the present invention is based upon the principle of inducing a stimulus of tissue regeneration through the exposure to specific electromagnetic stresses. For example, the resonance at a cellular level can induce regeneration of tissue lesions, i.e., induce or accelerate the reparative processes. In general, the invention can be applied to all the lesions that can be repaired through biological processes that can be stimulated through electromagnetic resonance. Experiments have shown that the most significant effectiveness is obtained for chronic lesions, the ones that represent the most serious consequences of complex syndromes, such as diabetes, and the start of a degenerative cascade that is very difficult to stop through systemic therapy.

In various embodiments, the apparatus is configured for generating an electromagnetic wave with specific contributions in frequency that is constituted, as a cascade, by pulses generated at a certain frequency.

In particular, in various embodiments, a given number of these base pulses or bursts (in a typical range of from 2 to 200 pulses) is generated in a “packet”.

In various embodiments, the packets in turn are generated in a cascade of a certain number of these packets, the frequency of which enables generation of a “train” of packets.

The frequency content of these pulses, packets, and trains is hence equivalent to obtaining the resonance frequencies, and those alone, that characterize the typical ranges of the various organs/tissues. Moreover, this structure of the signal enables not only the desired frequencies and only those to be obtained, but also enables their modification on one and the same apparatus in a simple and deterministic way, by adjusting the typical parameters of the components of the wave.

In various embodiments, this process of construction of the signal can be extended to further levels beyond the wave trains (i.e., constructing “sets of trains” and so forth, with a construction at progressively higher levels) in the case where it were to desired to obtain a further capacity of regulation of the frequency contents within specific and predetermined ranges. In this way, it is in fact possible to insert in the resulting signal all and only the “typical” frequencies of the target structures, only within the effective ranges, and with the capacity of modifying them in a very convenient way, in order to modify or rather pinpoint the therapeutic “targets” to be achieved simultaneously.

In various embodiments, to obtain the optimal therapeutic effect and the necessary capacity of regulation of the apparatus in its frequency contents, the bursts, i.e., the base elements on which the entire signal is constructed, can have different waveforms, such as, for example, a sinusoidal, a square-wave, or a triangular waveform.

In various embodiments, also the bursts themselves can contain some spikes, which guarantee that, in addition to the main frequency, there is an additional adequate content of harmonics.

The present invention represents an important innovation in the field of the treatment of lesions of human and animal tissues, in particular, but not exclusively, of skin ulcers and even more in particular the ones consequent on peripheral arthery disease (PAD), such as for example those of the so-called “diabetic foot”. The apparatus according to the invention can be used, for example, in the field of flebology for the treatment of effusions, thrombophlebitis, lymphopathy with oedema, bedsores, post-radiotherapy ulcers, and haemorrhoids. The invention also finds application in the field of orthopaedics and rheumatology for the treatment of arthrosis and pseudoarthrosis, for consolidation of fractures, and the treatment of carpal tunnel syndrome, tendinitis, enthesitis, fasciitis, capsulitis, arthritis and periarthritis, meniscopathy, discopathy, neuropathy, low-back pain/sciatica, cervical pain, myalgia in general, osteoporosis, disk protrusion and herniation, pathological conditions of the cartilage, osteochondritis, etc. In the field of sports medicine, the apparatus according to the invention can be used for the treatment of epicondylitis, epitrocleitis, various muscular traumas, distorsions with and without ligament lesions, cramp, gonalgia, pubalgia, etc.

### BRIEF DESCRIPTION OF THE DRAWINGS

The present invention will now be described in detail with reference to the attached drawings, which are provided purely by way of non-limiting example and in which:

FIGS. 1a to 1e show different waveforms for the base pulses;

FIG. 2 shows the composition of a packet comprising a plurality of base pulses according to FIG. 1;

FIG. 3 shows the composition of a train of packets comprising a plurality of packets according to FIG. 2;

FIG. 4 shows a set of trains of packets according to FIG. 3;

FIG. 5 is a block diagram of an embodiment of an apparatus for therapeutic treatment according to the invention;

FIGS. 6 to 7c show possible embodiments of antennas for the apparatus for therapeutic treatment according to the invention; and

FIGS. 8a to 9b show embodiments of signals generated via the apparatus for therapeutic treatment according to the invention.

### DETAILED DESCRIPTION OF EMBODIMENTS

Illustrated in the ensuing description are various specific details aimed at an in-depth understanding of the embodiments. The embodiments can be obtained without one or more of the specific items, or with other methods, components, materials, etc. In other cases, known structures, materials, or operations are not shown or described in detail so that various aspects of the embodiments will not be obscured.

The reference to “an embodiment” or “one embodiment” in the framework of this description is intended to indicate that a particular configuration, structure, or characteristic described in relation to the embodiment is comprised in at least one embodiment. Hence, phrases such as “in an embodiment” or “in one embodiment” that may be present in various points of this description do not necessarily refer to one and the same embodiment. Moreover, particular conformations, structures, or characteristics can be combined in an adequate way in one or more embodiments.

The references appearing herein are used only for convenience and hence do not define the sphere of protection or the scope of the embodiments.

As mentioned previously, the apparatus according to the present invention is configured for generating an electromagnetic wave with specific contributions in frequency.

In particular, the inventor has found that the signal generated via the apparatus of the present invention must present specific contributions in the spectrum between a minimum frequency fₘᵢₙ and a maximum frequency fₘₐₓ.

In addition, the inventor has found that the intensity of the electromagnetic wave can be low in said range. For example, it is typically sufficient for the intensity of the magnetic field in said frequency range to be below the intensity of the Earth's magnetic field (i.e., below 40 μT).

In various embodiments, to generate said electromagnetic field, the apparatus is configured for generating a signal that is made up as a cascaded of pulses generated at a certain frequency.

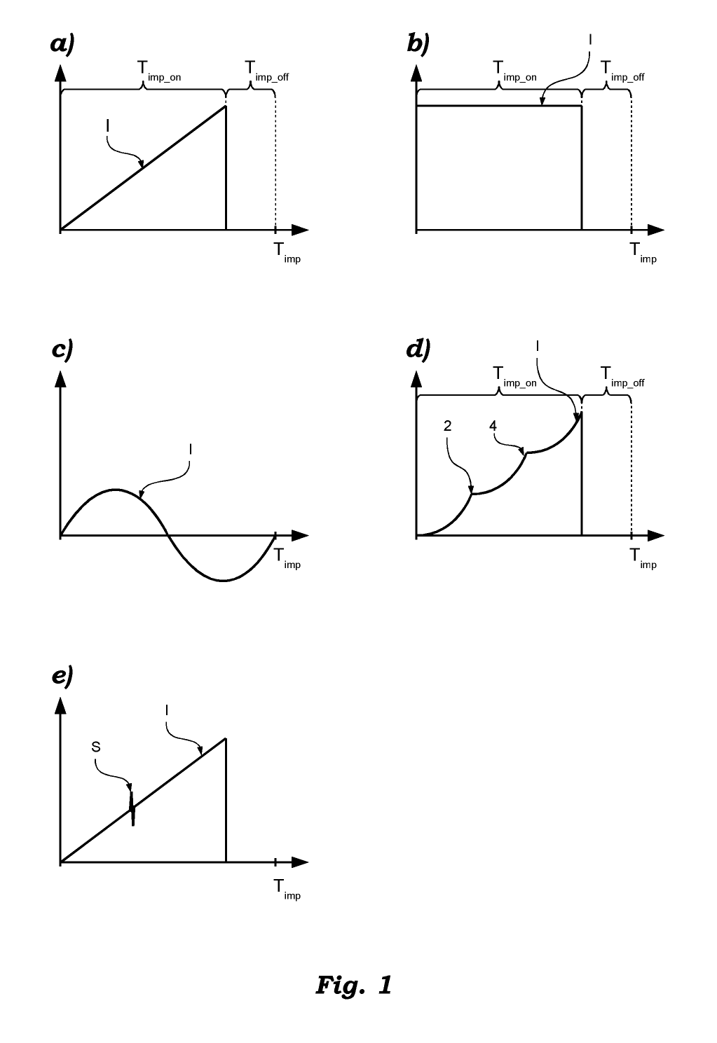

FIG. 1 illustrates possible waveforms of such a base pulse I.

For example, in the embodiment considered, the base pulse I can be a sawtooth or triangular waveform (FIG. 1a), a square waveform (FIG. 1b), or a sinusoidal waveform (FIG. 1c). The person skilled in the art will appreciate that said pulse I may also have other waveforms. For example, FIG. 1d shows a base pulse I comprising a series of three curved profiles in such a way that the waveform is increasing and comprises two cusps 2 and 4.

In various embodiments, a pulse-width modulation (PWM) can be applied to said base pulse I with a duration Tᵢₘₚ. Consequently, for a duration Tᵢₘₚ^{^{—}}ₒₙ the pulse is activated and for a duration Tᵢₘₚ^{^{—}}_{off} the pulse is deactivated, where the durations Tᵢₘₚ^{^{—}}ₒₙ and Tᵢₘₚ^{^{—}}_{off} can be varied in the interval ]0; Tᵢₘₚ[.

In various embodiments, the base pulse I has a frequency fᵢₘₚ of between 100 Hz and 1 kHz, preferably between 100 and 400 Hz, even more preferably between 150 and 250 Hz. For example, in one embodiment, the base pulse I has a frequency fᵢₘₚ of 212.72 Hz, i.e., the duration of a single pulse Tᵢₘₚ is 4.7 ms.

In various embodiments, a given number of these base pulses I are grouped together to generate a “packet” P.

In a substantially similar way, a pulse-width modulation can be applied also to said packet P; i.e., the base pulses I within a packet P with a duration T_{pac} are activated for a duration T_{pac}^{^{—}}ₒₙ and deactivated for a duration T_{pac}^{^{—}}_{off}, where the durations T_{pac}^{^{—}}ₒₙ and T_{pac}^{^{—}}_{off} can be varied in the interval ]0; T_{pac}[.

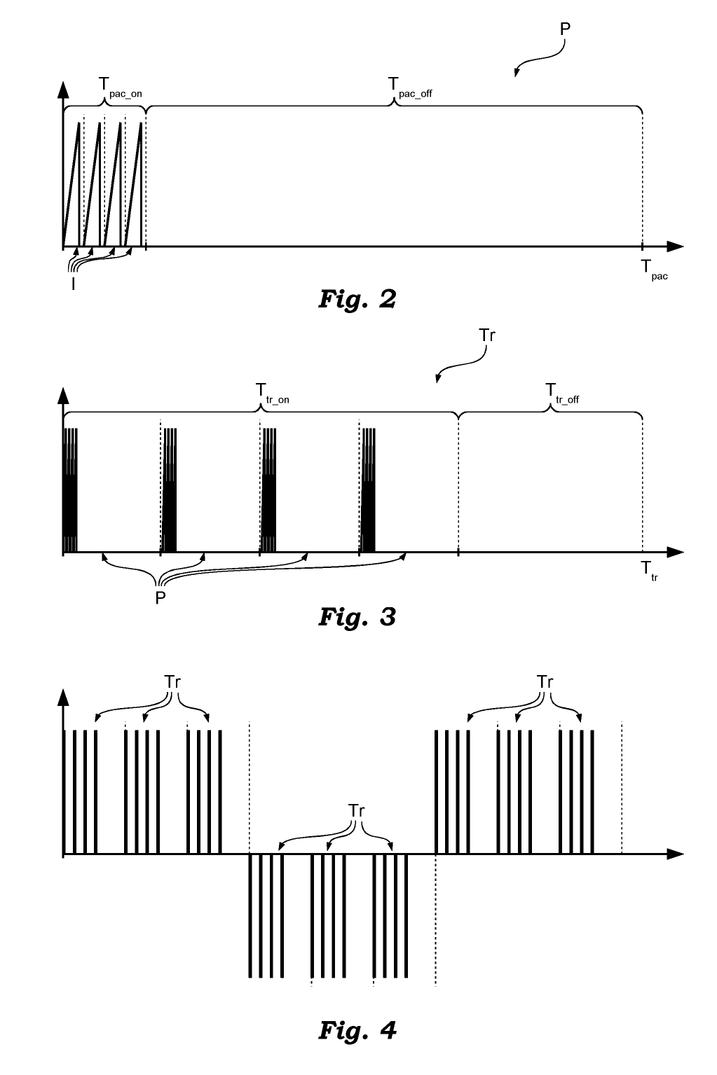

For example, FIG. 2 shows an embodiment of a packet P comprising four base pulses I.

In one embodiment, the duration of the packet T_{pac} is between 5 ms and 2 s, preferably between 20 ms and 1 s, even more preferably between 25 ms and 500 ms.

For instance, in the case where the duration of the packet T_{pac} is 1/3 s=333.33 ms, the frequency of repetition of the packet f_{pac} would be 3 Hz. Consequently, said packet P could contain up to 70 pulses with a duration of 4.7 ms. In particular, in the case where said packet contained only four base pulses I, the duration T_{pac}^{^{—}}ₒₙ would be 18.8 ms and the duration T_{pac}^{^{—}}_{off} would be 314.53 ms.

The inventor has found that usually it is expedient to size the frequency fᵢₘₚ and the duration T_{pac} in such a way that a packet P can contain between 2 and 200 base pulses I.

In various embodiments, a given number of these packets P are grouped for generating a “train of packets” Tr.

Also in this case it is possible to apply to the train Tr a pulse-width modulation, i.e., the packets P within a train Tr with a duration Tₜᵣ are activated for a duration Tₜᵣ^{^{—}}ₒₙ and deactivated for a duration Tₜᵣ^{^{—}}_{off}, where the durations Tₜᵣ^{^{—}}_{on and T}ₜᵣ^{^{—}}_{off} can be varied in the interval ]0; Tₜᵣ[.

For example, FIG. 3 shows an embodiment of a train Tr comprising four packets P.

In one embodiment, the duration of the train Tₜᵣ is between 25 ms and 10 s, preferably between 100 ms and 5 s, even more preferably between 1 and 5 s.

For example, in the case where the duration of the train Tₜᵣ is 3.80 s, the frequency of repetition of a train fₜᵣ would be 0.2632 Hz. Consequently, said train Tr could contain up to 11 packets with a duration of 333.33 ms. In particular, in the case where the packet contained 10 packets P, the duration Tₜᵣ^{^{—}}ₒₙ would be 3.33 s and the duration Tₜᵣ^{^{—}}_{off} would be 0.47 s.

The inventor has found that it is usually expedient to size the durations Tₜᵣ and T_{pac} in such a way that a train can contain between 2 and 100 packets P.

Consequently, the final signal comprises a series of base pulses I that are activated and deactivated according to the timing characteristics defined via the durations T_{pac}^{^{—}}ₒₙ, T_{pac}^{^{—}}_{off}, Tₜᵣ^{^{—}}ₒₙ and Tₜᵣ^{^{—}}_{off}. Consequently, in the case where the signal were to remain always activated (i.e., T_{pac}^{^{—}}_{off}=0 and Tₜᵣ^{^{—}}_{off}=0) the spectrum of the signal would contain only the spectrum of the base pulse I. For example, in the case of a base pulse I with sinusoidal waveform at 212.72 Hz, the spectrum would contain a single peak at 212.72 Hz. However, since the periodic signal is truncated remaining defined only within a certain interval of definition, the resulting spectrum is broadened in the frequency domain by a value equal to the inverse of the interval of definition of the signal itself. Consequently, the final signal also comprises the characteristics in frequency of the packets P and of the trains T, i.e., the harmonics for the frequencies f_{pac} and fₜᵣ.

In fact, the inventor has found that in this way it is possible to define via the duration Tₜᵣ a minimum frequency and via the duration T_{pac} a maximum frequency. Consequently, the apparatus described herein stimulates the cells not via the fundamental harmonics of the base pulse I but via the secondary harmonics resulting in the interval between fₜᵣ and f_{pac}; i.e., the frequency of the train fₜᵣ corresponds to the minimum frequency fₘᵢₙ, and the frequency of the packet f_{pac} corresponds to the maximum frequency fₘₐₓ.

In fact, the inventor has found that, in the case where the harmonics are chosen to correspond to the “typical” frequencies of the target structures, these harmonics with low amplitude are sufficient for stimulating the target in an effective way.

In fact, the inventor has found that for the human body it is typically sufficient for the apparatus to simulate the target above all in the interval between 0.1 and 25.9 Hz. Moreover, experiments have shown that the maximum effectiveness can be obtained in the case where the frequency of the packet f_{pac} is chosen between 2.89 and 21.85 Hz and the frequency of the train fₜᵣ is chosen between 0.3 and 2.8 Hz and the frequency of repetition of the sets of trains between 0.1 and 0.3 Hz.

In general, the inventor has found that it is possible to vary the number of the harmonics and the characteristic profile of the spectrum of the signal between the frequencies fₜᵣ and f_{pac} by modifying principally the profile of the base pulse I, the number of pulses within a packet, and the number of packets P.

For example, the inventor has found that usually each packet P within a train T generates a peak in the interval between fₜᵣ and f_{pac}.

Moreover, also the base pulses I can contain some spikes, which guarantee that in addition to the main frequency there is an adequate content of additional harmonics.

For example, FIG. 1e shows an embodiment of a base pulse I having a sawtooth shape that comprises a spike S.

Finally, in various embodiments, this process of construction of the signal can be extended to further levels beyond the wave trains (i.e., constructing “sets of trains” and so forth, with a construction at progressively higher levels) in the case where it were desired to obtain a further capacity of regulation of the frequency contents within specific and predetermined ranges.

For example, FIG. 4 shows an embodiment in which three trains Tr are grouped to form a set of trains. FIG. 4 shows also that the polarity of said set of trains can be alternated, i.e., reversed for each successive set of trains.

In this way, in fact, it is possible to “insert in the resulting signal” all and only the “typical” frequencies of the target structures, only within the effective ranges, and with the capacity of modifying them in a very convenient way, in order to modify or rather pinpoint the therapeutic “targets” to be achieved simultaneously.

The frequency content of these pulses, packets, and trains is hence equivalent to obtaining the resonance frequencies, and those alone, that characterize the typical range of the various organs/tissues. Moreover, this structure of the signal enables not only the desired frequencies to be obtained and only those, but also enables modification thereof on one and the same apparatus in a simple and deterministic way, adjusting the typical parameters of the components of the wave.

Moreover, it appears that the reversal of polarity of the signal will enable an effective renewal of the conditions of the overall state of the electric potential that characterizes the cell membrane and its correct metabolic and biochemical behaviour. In general, said reversal of polarity of the pulses I can be made after given time intervals, hence also at the level of pulses, packets, and/or trains. However, experiments have shown that the maximum effectiveness can be obtained in the case where said reversal of polarity is made between 80 and 200 s, preferably between 100 and 180 s, more preferably every 120 or 180 s.

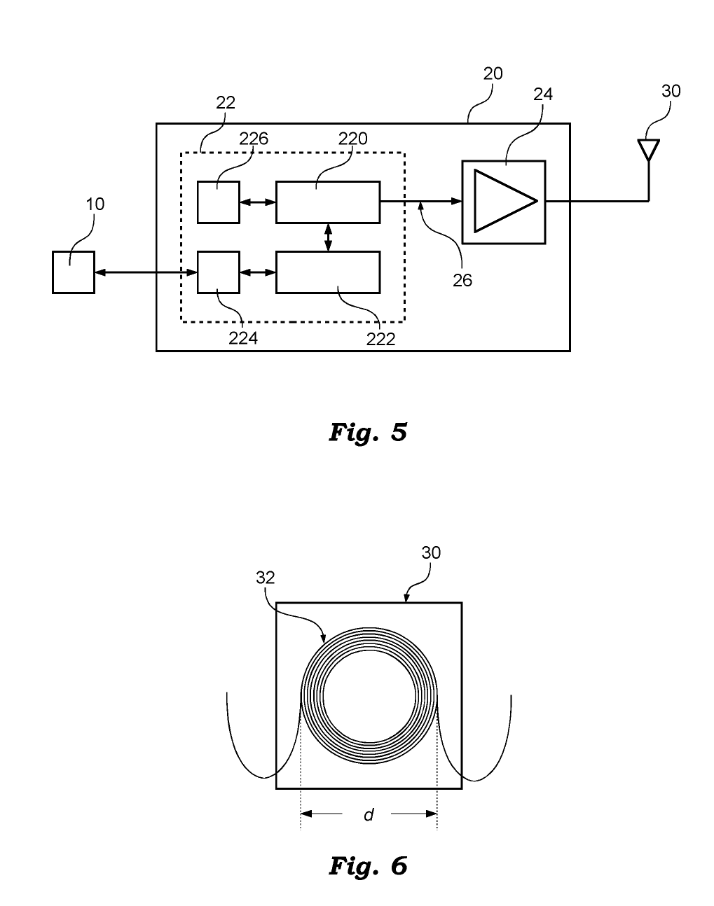

FIG. 5 shows a possible embodiment of the apparatus 20 for therapeutic treatments.

In the embodiment considered, the apparatus 20 comprises a control circuit 22 configured for generating a signal 26 that corresponds to the signal described previously, i.e., a signal comprising a plurality of base pulses I grouped into packets P and trains Tr. Said signal 26 is sent through a power amplifier 24 to an antenna 30.

In the embodiment considered, the control circuit 22 comprises a processing unit 220, such as for example a microcontroller, a DSP (Digital Signal Processor) or an FPGA (Field Programmable Gated Array), configured for generating the signal 26.

For example, in the embodiment considered, the control circuit 22 comprises a memory 222, such as, for example, an EEPROM (Electrically Erasable Programmable Read-Only Memory) or a FLASH memory, in which the characteristic data of the signal 26 are saved, such as for example values that identify the duration T_{pac}^{^{—}}ₒₙ, T_{pac}^{^{—}}_{off}, Tₜᵣ^{^{—}}ₒₙ and Tₜᵣ^{^{—off}}.

In the case where also the base pulses I are configurable, there may also be saved data that identify the waveform and/or the durations Tᵢₘₚ^{^{—}}ₒₙ and Tᵢₘₚ^{^{—}}_{off} (see FIG. 1).

For example, in one embodiment, the control circuit 22 comprises a waveform generator 226 configured for generating different waveforms (see for example FIG. 1) with a certain frequency fᵢₘₚ, and the processing unit 220 can be configured for activating and deactivating the signal coming from the waveform generator 226 via an electronic switch according to the durations T_{pac}^{^{—}}ₒₙ, T_{pac}^{^{—}}_{off}, Tₜᵣ^{^{—}}ₒₙ and Tₜᵣ^{^{—}}_{off}, and possibly also of the durations Tᵢₘₚ^{^{—}}ₒₙ and Tᵢₘₚ^{^{—}}_{off}.

In one embodiment, the characteristic data of the signal 26 are modifiable.

For example, in the embodiment considered, the control circuit 22 comprises a communication interface 224 for receiving the characteristic data of the signal 26 from an external configuration unit 10, such as for example a PC. For instance, said communication interface 224 can be an RS-232, USB (Universal Serial Bus) interface, or also LAN (Local Area Network) or WAN (Wide Area Network) network-interface cards for wired or wireless communication.

In one embodiment, the memory 222 comprises a plurality of profiles of treatment programs, where each program can present different characteristic data for the signal 26. In this case, the apparatus 20 also comprises a user interface that enables selection of the desired treatment program.

As mentioned previously, in one embodiment, the antenna 30 is a solenoid. In this case, the apparatus 20, in particular the amplifier 24, can be configured for driving the antenna 30 with a control in current. For example, typically it is sufficient to drive the antenna 30 with a current having a maximum amplitude of the base pulse I that can be set between 150 mA and 1 A, where an amplitude of 450 mA is typically used. In this case, it can also be envisaged that for each treatment program there can be set the amplitude of the signal to be sent to the antenna 30. For instance, said amplitude can be modified by appropriately setting the coefficient of amplification of the amplifier 24.

FIG. 6 shows in this context a possible embodiment of the antenna 30.

In the embodiment considered, the antenna 30 comprises a plurality of turns 32 of a conductor with external insulation. For example, in one embodiment a unipolar cable of copper or copper-silver with a diameter of 1 mm is used.

In the embodiment considered, the external diameter d of the solenoid 30 is comprised between 21 and 24 cm.

In one embodiment, the number of turns of the antenna 30 is a multiple of three, and preferably comprised between 3 and 72 turns.

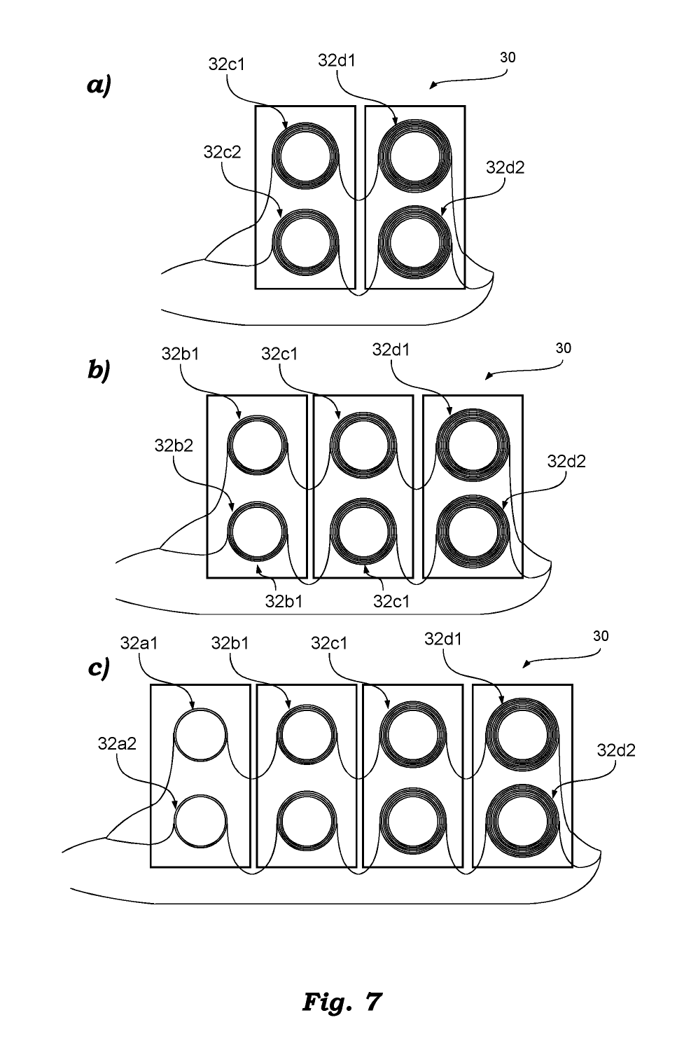

FIGS. 7a to 7c illustrate also the fact that the antenna 30 can comprise a plurality of these solenoids connected together.

For example, four solenoids 32c1, 32c2, 32d1, and 32d2 are connected together in FIG. 7a. In particular, in the embodiment considered, the solenoids 32c1 and 32d1 are connected in series to form a first set, and the solenoids 32c2 and 32d2 are connected in series to form a second set. In the embodiment considered, said sets are connected in parallel.

Moreover, FIG. 7a shows also the fact that said solenoids (32c1, 32c2, 32d1, and 32d2) can have a different number of turns. For example, in the embodiment considered, the solenoids 32c1 and 32c2 and the solenoids 32d1 and 32d2 have in each set the same number of turns, where the number of turns of the solenoids 32d1 and 32d2 is greater than the number of the solenoids 32c1 and 32c2.

FIGS. 7b and 7c show that said type of connection can also be extended respectively to six (32b1, 32b2, 32c1, 32c2, 32d1, and 32d2) or eight solenoids (32a1, 32a2, 32b1, 32b2, 32c1, 32c2, 32d1, and 32d2).

In general, the antenna 30 can then comprise a first set of solenoids, where the solenoids have numbers of turns, respectively, equal to the numbers of turns of the corresponding solenoids of the second set, and where the solenoids of the first set are connected in series.

In one embodiment, the antenna 30 also comprises a second set of solenoids, where the solenoids of the second set are connected in series.

In one embodiment, the first set and the second set are connected in parallel.

In one embodiment, the number of the solenoids of the first set is equal to the number of the solenoids of the second set.

In one embodiment, each solenoid of the first set has a number of turns that corresponds to the number of turns of a respective solenoid of the second set. Consequently, the antenna 30 comprises once again two solenoids with a certain number of turns, where the first forms part of the first set and the second forms part of the second set.

The person skilled in the art will appreciate that there can be used also other antennas 30 and/or that control of the antenna 30 can be performed in voltage.

In one embodiment, the apparatus comprises a treatment program for simulation of the delta waves of the human brain.

The inventor has found that the delta waves are typically comprised between 0.4 Hz and 3 Hz and comprise four characteristic peaks.

Consequently, in a possible embodiment, the minimum frequency fₘᵢₙ of the signal 26, i.e., the frequency of the trains fₜᵣ, is set at 0.4 Hz, and the maximum frequency fₘₐₓ, i.e., the frequency of the packets f_{pac}, is set at 2.89 Hz.

In the embodiment considered, the duration of a packet T_{pac} is 1/2.89 s=346.0 ms, and the duration of a train Tₜᵣ is 1/0.4 s=2.5 s.

Moreover, in a possible embodiment, to create the four characteristic peaks, each train Tr of the signal 26 comprises four packets P.

Consequently, the duration Tₜᵣ^{^{—}}ₒₙ is 4×1/2.89 s=1.384 s and the duration Tₜᵣ^{^{—}}_{off} is 1.116 s.

Finally, the inventor has found that, to obtain the characteristic profile in frequency of the delta waves, it is appropriate to use 44 sawtooth base pulses I with a frequency fᵢₘₚ of 212.72 Hz for each packet P and reverse the polarization of the trains every 120 s for a duration of treatment of 8 minutes.

Consequently, in the embodiment considered, the duration T_{pac}^{^{—}}ₒₙ is 44×1/212.72 s=206.8 ms, and the duration T_{pac}^{^{—}}_{off} is 139.2 ms.

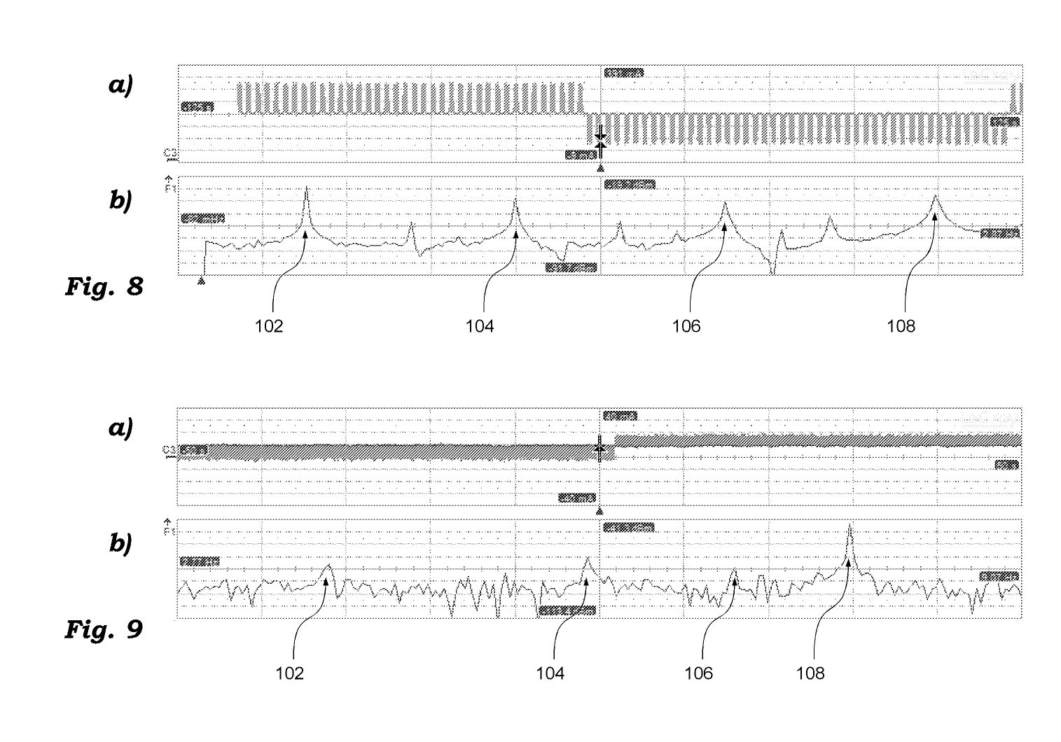

FIG. 8 shows in this context the signal 26 (FIG. 8a) and a representation of the spectrum of the signal in the interval between 0 Hz and 2.87 Hz (FIG. 8b). In particular, there may be noted the presence of four characteristic peaks: 102, 104, 106, and 108.

In one embodiment, the apparatus comprises a treatment program for the simulation of the theta waves of the human brain.

The inventor has found that the theta waves are typically comprised between 1.94 Hz and 7.9 Hz and comprise four characteristic peaks.

Consequently, in a possible embodiment, the minimum frequency fₘᵢₙ of the signal 26, i.e., the frequency of the trains fₜᵣ, is set at 1.94 Hz, and the maximum frequency fₘₐₓ, i.e., the frequency of the packets f_{pac}, is set at 7.9 Hz.

In the embodiment considered, the duration of a packet T_{pac} is 1/7.9 s=126.6 ms, and the duration of a train Tₜᵣ is 1/1.94 s=515.5 ms.

Moreover, in a possible embodiment, to create the four characteristic peaks, each train Tr of the signal 26 comprises four packets P.

Consequently, the duration Tₜᵣ^{^{—}}ₒₙ is 4×1/7.9 s=506.3 ms, and the duration Tₜᵣ^{^{—}}_{off} is 9.1 ms.

Finally, the inventor has found that, to obtain the characteristic profile in frequency of the theta waves, it is expedient to use four sawtooth base pulses I with a frequency fᵢₘₚ of 212.72 Hz for each packet P and reverse the polarization of the trains every 120 s.

Consequently, in the embodiment considered, the duration T_{pac}^{^{—}}ₒₙ is 4×1/212.72 s=18.8 ms, and the duration T_{pac}^{^{—}}_{off is} 107.8 ms.

FIG. 9 shows in this context the signal 26 (FIG. 9a) and a representation of the spectrum of the signal in the interval between 0 Hz and 10.65 Hz (FIG. 9b). In particular, there may be noted the presence of four characteristic peaks 102, 104, 106, and 108.

In one embodiment, the apparatus comprises the following treatment programs, which can be present also individually or in groups for carrying out a specific therapeutic treatment:

1) program 1: saw-tooth base pulse (see FIG. 1a), where the duration of the base pulse is Tᵢₘₚ=4.7 ms, the number of base pulses in a pulse packet is 44, the pause between the packets is T_{pac}^{^{—}}_{off}=140 ms, the number of pulse packets in a pulse train is 4, and the pause between the trains is Tₜᵣ^{^{—}}_{off}=1450 ms;

2) program 2: saw-tooth base pulse, where the duration of the base pulse is Tᵢₘₚ=4.32 ms, the number of base pulses in a pulse packet is 34, the pause between the packets is T_{pac}^{^{—}}_{off}=105 ms, the number of pulse packets in a pulse train is 4, and the pause between the trains is Tₜᵣ^{^{—}}_{off}=1100 ms;

3) program 3: saw-tooth base pulse, where the duration of the base pulse is Tᵢₘₚ=4.7 ms, the number of base pulses in a pulse packet is 20, the pause between the packets is T_{pac}^{^{—}}_{off}=55 ms, the number of pulse packets in a pulse train is 4, and the pause between the trains is Tₜᵣ^{^{—}}_{off}=600 ms;

4) program 4: saw-tooth base pulse, where the duration of the base pulse is Tᵢₘₚ=4.32 ms, the number of base pulses in a pulse packet is 18, the pause between the packets is T_{pac}^{^{—}}_{off}=37 ms, the number of pulse packets in a pulse train is 4, and the pause between the trains is Tₜᵣ^{^{—}}_{off}=480 ms;

5) program 5: saw-tooth base pulse, where the duration of the base pulse is Tᵢₘₚ=4.32 ms, the number of base pulses in a pulse packet is 16, the pause between the packets is T_{pac}^{^{—}}_{off}=24 ms, the number of pulse packets in a pulse train is 4, and the pause between the trains is Tₜᵣ^{^{—}}_{off}=380 ms;

6) program 6: saw-tooth base pulse, where the duration of the base pulse is Tᵢₘₚ=4.32 ms, the number of base pulses in a pulse packet is 14, the pause between the packets is T_{pac}^{^{—}}_{off}=16 ms, the number of pulse packets in a pulse train is 4, and the pause between the trains is Tₜᵣ^{^{—}}_{off}=350 ms;

7) program 7: saw-tooth base pulse, where the duration of the base pulse is Tᵢₘₚ=4.7 ms, the number of base pulses in a pulse packet is 12, the pause between the packets is T_{pac}^{^{—}}_{off}=6.6 ms, the number of pulse packets in a pulse train is 4, and the pause between the trains is Tₜᵣ^{^{—}}_{off}=220 ms;

8) program 8: saw-tooth base pulse, where the duration of the base pulse is Tᵢₘₚ=4.32 ms, the number of base pulses in a pulse packet is 10, the pause between the packets is T_{pac}^{^{—}}_{off}=2.55 ms, the number of pulse packets in a pulse train is 4, and the pause between the trains is Tₜᵣ^{^{—}}_{off}=176 ms; and

9) program 9: base pulse with cusps (see FIG. 1d), where the duration of the base pulse is Tᵢₘₚ=5.27 ms, the number of base pulses in a pulse packet is 5, the pause between the packets is T_{pac}^{^{—}}_{off}=36 ms, the number of pulse packets in a pulse train is 20, and the pause between the trains is Tₜᵣ^{^{—}}_{off}=3000 ms.

Consequently, the programs listed above create the following frequencies:

1) program 1: the frequency of the base pulses is fᵢₘₚ=212.76 Hz, the frequency of the pulse packets is f_{pac}=2.89 Hz, and the frequency of the pulse train is fₜᵣ=0.4 Hz;

2) program 2: the frequency of the base pulses is fᵢₘₚ=231.48 Hz, the frequency of the pulse packets is f_{pac}=3.98 Hz, and the frequency of the pulse train is fₜᵣ=0.5 Hz;

3) program 3: the frequency of the base pulses is fᵢₘₚ=212.76 Hz, the frequency of the pulse packets is f_{pac}=6.71 Hz, and the frequency of the pulse train is fₜᵣ=0.9 Hz;

4) program 4: the frequency of the base pulses is fᵢₘₚ=231.48 Hz, the frequency of the pulse packets is f_{pac}=8.71 Hz, and the frequency of the pulse train is fₜᵣ=1.1 Hz;

5) program 5: the frequency of the base pulses is fᵢₘₚ=231.48 Hz, the frequency of the pulse packets is f_{pac}=10.73 Hz, and the frequency of the pulse train is fₜᵣ=1.4 Hz;

6) program 6: the frequency of the base pulses is fᵢₘₚ=231.48 Hz, the frequency of the pulse packets is f_{pac}=13.07 Hz, and the frequency of the pulse train is fₜᵣ=1.6 Hz;

7) program 7: the frequency of the base pulses is fᵢₘₚ=212.76 Hz, the frequency of the pulse packets is f_{pac}=15.87 Hz, and the frequency of the pulse train is fₜᵣ=2.1 Hz;

8) program 8: the frequency of the base pulses is fᵢₘₚ=231.48 Hz, the frequency of the pulse packets is f_{pac}=21.85 Hz, and the frequency of the pulse train is fₜᵣ=2.8 Hz; and

9) program 9: the frequency of the base pulses is fᵢₘₚ=189.75 Hz, the frequency of the pulse packets is f_{pac}=16.03 Hz, and the frequency of the pulse train is fₜᵣ=2.8 Hz.

For example, given the temporal characteristics described previously, the programs can create the characteristic frequencies listed below, i.e., main peaks in the spectrum comprised in the range between the minimum frequency and the maximum frequency (for simplicity only the peaks that exceed a certain power threshold are listed):

1) program 1: 0.4 and 2.89 Hz;

2) program 2: 0.5 and 3.98 Hz;

3) program 3: 0.9 and 6.71 Hz;

4) program 4: 1.1 and 8.71 Hz;

5) program 5: 1.4, 9.2 and 10.73 Hz;

6) program 6: 1.6, 12.20 and 13.07 Hz;

7) program 7: 2.1 and 15.87 Hz;

8) program 8: 2.8, 8.6, 14 and 21.85 Hz; and

9) program 9: 2.8, 15.8 and 16.03 Hz.

As may be noted, each signal comprises one peak that corresponds to the frequencies of the packets and one peak that corresponds to the frequencies of the trains.

In various embodiments, the duration of the treatment for all these programs is 480 s.

In various embodiments, the polarity of the programs 1 to 8 is reversed every 120 s, whilst the polarity of the program 9 is reversed every 180 s.

The inventor has noted that the programs mentioned above stimulate the parts of the body and/or generate the effects in the human body listed below:

1) program 1: central nervous system (CNS), limiting its function as far as creating sub-hypnotic states; ansiolytic, sedative, hypno-inducing effect;

2) program 2: paranasal sinuses and cranial sinuses, bronchia and respiratory tree, generalized organic stimulus; improvement of pulmonary ventilation;

3) program 3: CNS and peripheral nervous system, stimulation, and stimulating and repairing effect;

4) program 4: neurovegetative system and correlated functions;

5) program 5: system of metabolization of endogenous and exogenous substances, liver, lungs, stomach; anti-inflammatory and disintoxicating effect in support of pharmacological therapies in progress and release of states of homotoxicological deposit;

6) program 6: artero-venous and lymphatic circulatory system;

7) program 7: synovial membranes, articular capsules, tendons, cartilage, and mediators involved in flogosis; anti-inflammatory and antalgic effect;

8) program 8: musculoskeletal apparatus and mediators involved in generation of pain, including the production of substance P; antalgic effect;

9) program 9: psychological system, neurological system, endocrine system, immunitary stimulation; regulating and cell-regenerating effect.

In various embodiments, the programs mentioned above are combined for treating certain pathological conditions.

For example, in one embodiment, for treating a diabetic foot, a sequence is used that comprises in order program 9, program 6, and program 8; namely, for the indicated duration of a program of 480 s, the duration of the entire treatment would be 1440 s.

In one embodiment, for the treatment of bone fractures, a sequence is used that comprises in order program 6 and program 8; namely, for the indicated duration of a program of 480 s, the duration of the entire treatment would be 960 s.

In one embodiment, for the treatment of osteoporosis, a sequence is used that comprises in order program 8 and program 9; namely, for the indicated duration of a program of 480 s, the duration of the entire treatment would be 1440 s.

In various embodiments, to facilitate use by the user, the sequences of the treatment programs can also be stored as distinct programs.

As mentioned previously, it is not necessary to save all nine programs in the memory of the device. For example, in a device exclusively dedicated to the treatment of a diabetic foot there could be saved only the programs 6, 8 and 9, and the device could reproduce automatically the corresponding sequence of treatment programs.

Moreover, as mentioned previously, the characteristic data of the signal 26 may also be provided via an external configuration unit 10. For example, in one embodiment, the communication interface 224 is a reader of an exchangeable memory, such as a USB drive or a memory card, such as a SD or MMC memory card, on which are stored:
the characteristic data of at least one treatment program, such as:

a) the base pulse type,

b) the base pulse duration or the base pulse frequency,

c) the number of base pulses in a pulse packet,

d) the pause between the packets, the packet duration or the pulse packet frequency,

e) the number of pulse packets in a pulse train,

f) the pause between the pulse trains, the pulse train duration or the pulse train frequency,
the signal amplitude, and/or
the sequence of treatment programs to be executed.

In place of the specific temporal characteristics, only the frequencies to be stimulated may be stored and the apparatus may calculate the respective temporal characteristic with the method described previously.

In this way, a doctor may adapt the operation of the apparatus to the needs of a specific patient.

For example, in one embodiment, a therapy protocol is stored on this memory, which defines e.g. the treatment days and hours, the treatment programs to be executed and the respective treatment durations.

Moreover, in one embodiment, the apparatus stores on this exchangeable memory a log file, which permits to analyze the sessions performed, such as the treatment days and time, the treatment programs used and/or the treatment durations actually effected.

In this case, the doctor may verify immediately, e.g. by means of an appropriate software program, if the therapy protocol has been observed.

Of course, without prejudice to the principle of the invention, the details of construction and the embodiments may vary widely with respect to what has been described and illustrated herein purely by way of example, without thereby departing from the scope of the present invention, as defined by the ensuing claims.

1. An apparatus for therapeutic treatment with electromagnetic waves comprising a control circuit configured for generating a signal to be transmitted to an antenna (30) for the generation of electromagnetic waves,
wherein said signal comprises a plurality of base pulses grouped in pulse packets and in pulse trains, where each pulse packet consists of a series of base pulses followed by a first pause, and where each pulse train consists of a series of pulse packets followed by a second pause,
said apparatus wherein said control circuit is configured for reversing the polarity of said base pulses after a given time interval.

2. The apparatus according to claim 1, wherein the frequency of said base pulses is between 100 Hz and 1 kHz, preferably between 100 and 400 Hz, preferably between 150 and 250 Hz.

3. The apparatus according to claim 1, wherein the frequency of repetition of said pulse packet is between 2.89 and 25.9 Hz.

4. The apparatus according to claim 1, wherein the frequency of repetition of said pulse trains is between 0.3 and 2.8 Hz.

5. The apparatus according to claim 1, wherein said time interval is between 80 and 200 s, preferably between 120 and 180 s.

6. The apparatus according to claim 1, wherein said signal comprises a plurality of trains grouped in sets of trains and in pulse trains, wherein each set of trains consists of a series of trains followed by a third pause, and wherein the frequency of repetition of said sets of trains is between 0.1 and 0.3 Hz.

7. The apparatus according to claim 1, wherein:
each base pulse has a sawtooth, square-wave, or sinusoidal waveform; or
each base pulse comprises a series of curved profiles in such a way that in a pulse time interval the waveform is increasing and comprises a plurality of cusps.

8. The apparatus according to claim 1, wherein each base pulse comprises at least one spike.

9. The apparatus according to claim 1, comprising a memory, saved in which are the characteristic temporal data of said pulse packets and said pulse trains for a plurality of treatment programs.

10. The apparatus according to claim 1, wherein said control circuit is configured for applying to said base pulse a pulse-width modulation.
